(19) United States
(12) Patent Application Publication
(10) Pub. No.: US 2026/0018288 A1
(43) Pub. Date: Jan. 15, 2026

(54) A COMPUTER-IMPLEMENTED METHOD OF DETERMINING IF A MEDICAL DATA SAMPLE REQUIRES REFERRAL FOR INVESTIGATION FOR A DISEASE

(71) Applicant: University of Liverpool, Liverpool (GB)

(72) Inventors: Yalin Zheng, Liverpool (GB); David Wong, Liverpool (GB); Mark Johnson, Liverpool (GB); Simon Harding, Liverpool (GB); Gabriela Czanner, Liverpool (GB); Dongxu Gao, Liverpool (GB); Wenyue Zhu, Liverpool (GB)

(21) Appl. No.: 18/994,127

(22) PCT Filed: Jul. 6, 2023
(86) PCT No.: PCT/GB2023/051791

§ 371 Date: Jan. 14, 2025

(30) Foreign Application Priority Data
Jul. 20, 2022 (GB) 2210624.9

(51) Int. Cl. G16H 50/20; G16H 30/40

(52) U.S. Cl. CPC G16H 50/20; G16H 30/40

(57) ABSTRACT

According to the subject-matter of the present disclosure, there is provided a computer-implemented method of determining if a medical data sample requires referral for investigation for a disease. The method comprises: performing a pairwise comparison of the medical data sample against each example of medical data in a reference data set using one or more machine learning algorithms to determine a difference in severity of the medical data compared to each example medical data in the reference data set; and flagging the medical data sample as requiring referral for investigation for the disease based on results of the pairwise comparisons, wherein the reference data set includes a plurality of medical data examples, the plurality of medical data examples ranked according to their degree of severity of disease

### FIELD

The subject-matter of the present disclosure relates to determining if medical data requires referral for investigation for a disease.

### BACKGROUND

Various medical conditions can be diagnosed by observing features in medical data. Typically, a diagnosis is made by a trained medical professional. In certain areas, e.g. those with high population density, there may be insufficient medical personnel who are trained to assess the high-volume of medical data from patients suspected as having a particular disease.

Artificial intelligence (AI) methods have been proposed with a binary output, e.g. to categorise medical data as having disease and not having disease. However, such methods have insufficient accuracy and flexibility.

It is an aim of the present subject-matter to alleviate such problems and improve on prior art methods.

### SUMMARY

According to the subject-matter of the present disclosure, there is provided a computer-implemented method of determining if a medical data sample requires referral for investigation for a disease, the method comprising: performing a pairwise comparison of the medical data sample against each example of medical data in a reference data set using one or more machine learning algorithms to determine a difference in severity of the medical data compared to each example medical data in the reference data set; and flagging the medical data sample as requiring referral for investigation for the disease based on results of the pairwise comparisons, wherein the reference data set includes a plurality of medical data examples, the plurality of medical data examples ranked according to their degree of severity of disease.

A machine learning algorithm is able to decide whether one image is more severe than another image with a higher degree of accuracy than trying to classify how severe a disease state is in an image directly

The computer-implemented method may further comprise: determining a position of the medical data sample against the medical data examples within the reference data set; and comparing the position of the medical data sample with a threshold for referral, wherein the flagging the medical data sample as requiring referral comprises flagging the medical data sample as requiring referral if the position of the medical data sample is above the threshold for referral.

The one or more machine learning algorithms may comprise one or more neural networks.

The or each neural network may be a convolutional neural network.

The one or more convolutional neural networks may be a plurality of convolutional neural networks.

Each of the plurality of convolutional neural networks may be trained on a different data set.

The computer-implemented method may further comprise amalgamating the results of the pairwise comparisons from each convolutional neural network, and wherein the sending the medical data sample for referral for investigation for the disease may be based on the amalgamated pairwise comparisons.

The amalgamating of the results of the pairwise comparisons may comprise supplying the results of the pairwise comparison as inputs to one or more lasso regression models, the or each lasso regression model may have a decision boundary associated with a threshold of disease severity.

The or each lasso regression model may be a plurality of lasso regression models and wherein the threshold of disease severity for each model is different.

The computer-implemented method may further comprise estimating a confidence interval of a probability of needing referral.

The estimating may comprise performing bootstrapping.

The amalgamating the results of the pairwise comparison may comprise accumulating results from each convolutional neural network, and comparing the accumulated results to the threshold for referral.

The amalgamating the results may comprise setting a plurality of thresholds of disease severity within the reference image set, and determining a frequency of occurrence of the medical data above each of the thresholds of disease severity.

The threshold for referral may correspond to one of the plurality of thresholds of disease severity.

The amalgamating the results of the pairwise comparison may comprise fitting an s-curve to the results of each convolutional neural network; determining a probability of requiring referral based each fitted S-curve; and performing linear discriminant analysis on the determined probabilities.

The computer-implemented method may further comprise selecting a subset of the plurality of convolutional neural networks using a selecting algorithm.

The selecting algorithm may comprise a lasso regression model, the lasso regression model having a decision boundary associated with the threshold for referral, the selecting may comprise applying the results from each convolutional neural network into the lasso regression model, ordering the convolutional neural networks in terms of accuracy at predicting referral, and selecting a predetermined number of the highest ranked convolutional neural networks as the subset.

The computer implemented method may further comprise: receiving the reference data set; and performing pairwise comparison of each medical data example of the plurality of examples of medical data within the reference data set against every other medical data example of the plurality of medical data example within the reference data set; and ranking the plurality of medical data examples according to a degree of severity of disease based on the pairwise comparisons.

The disease may be any of breast cancer, pneumonia, lung cancer, skin cancer, and cardiovascular disease.

The medical data may be any of a two-dimensional image, a three-dimensional image, or trace data.

The two-dimensional image may comprise any of X-ray and mammography X-ray, and/or wherein the three-dimensional image may comprise any of a magnetic resonance image, a computerised tomography images, or an Ultrasound image, and/or wherein the trace data comprises an eco-cardiogram.

According to an aspect of the present disclosure, there is provided a computer-implemented method of ranking a plurality of medical data examples within a reference data set, the method comprising: receiving the reference data set; and performing pairwise comparison of each medical data example within the reference data set against every medical data example within the reference data set; and ranking the plurality of medical data examples according to a degree of severity of disease based on the pairwise comparisons.

According to an aspect of the present disclosure, there is provided a transitory or non-transitory computer-readable medium including instructions stored thereon that when executed by one or more processors cause the processor to perform the method as claimed in any preceding claim.

### BRIEF DESCRIPTION OF DRAWINGS

The subject-matter of the present disclosure is best understood with reference to the accompanying figures, in which:

FIG. 1 shows a detailed flow chart of a computer-implemented method of categorising medical data as requiring further investigation for disease, according to an embodiment;

FIG. 2 shows a high-level flow diagram of pairwise comparison performed as part of the method from FIG. 1, according to an embodiment;

FIG. 3 shows a schematic of a convolutional neural network used for performing pairwise comparisons of medical data when performing the method from FIG. 2, according to an embodiment; and

FIG. 4 shows a flow chart of ranking medical data within a reference data set as part of the method from FIG. 1, according to an embodiment.

### DESCRIPTION OF EMBODIMENTS

All methods described herein may be computer-implemented methods, where each step is implemented on a computer. The computer may include a data store (or data storage), and one or more processors. The computer-implemented methods may be provided as instructions on a transitory, or non-transitory, computer-readable medium. The transitory or non-transitory computer-readable medium may be provided on the data store. The instructions, when executed by the one or more processors, configure the processor to perform the method steps described herein.

It will be appreciated that, whilst the following description describes one or more embodiments, features from different embodiments may be used in other embodiments without introducing new subject-matter that extends beyond the content of the present disclosure.

Diseases that may be classified using the methods described herein include breast cancer, pneumonia, lung cancer, skin cancer, and cardiovascular disease, and more. Those diseases, and others, can be subject to the methods described herein because they can be diagnosed using medical images (e.g. X-ray, mammograph X-ray, computerised tomography (CT) scan, magnetic resonance imaging (MRI), ultrasound scans, etc.), or using time series data (e.g. eco-cardiogram (ECG)). Time series data may be trace data. In this way, medical data may include medical images or trace data. In each case, the medical data includes physiological parameters of a subject.

For more details of the conditions that may be subject to the methods described herein, the classification can be performed on mammography X-rays to classify the specimen as benign or malignant. X-rays may be used to classify specimens as having pneumonia or normal, and may even be able to provide an indication as to what the underlying cause is, e.g. Covid-19. The same is true if CT scans are used to diagnose the patient. Pathology techniques using images of slide samples can be used for conditions such as breast cancer. Images of biopsies investigating for skin cancer can be subject to the methods described herein. The ECG waveforms can be used to classify the patient has having cardiovascular disease, or as normal.

Herein, the term “sample” may be used to mean an instance, e.g. one image, taken of a patient when investigating the patient for a particular disease. The term “example”, may be used herein to mean an instance, e.g. one image, within a data set, e.g. a reference data set, used for various purposes including training or comparison.

In one or more embodiments, there is provided a method of classifying if a disease is severe enough to require further investigation using medical images.

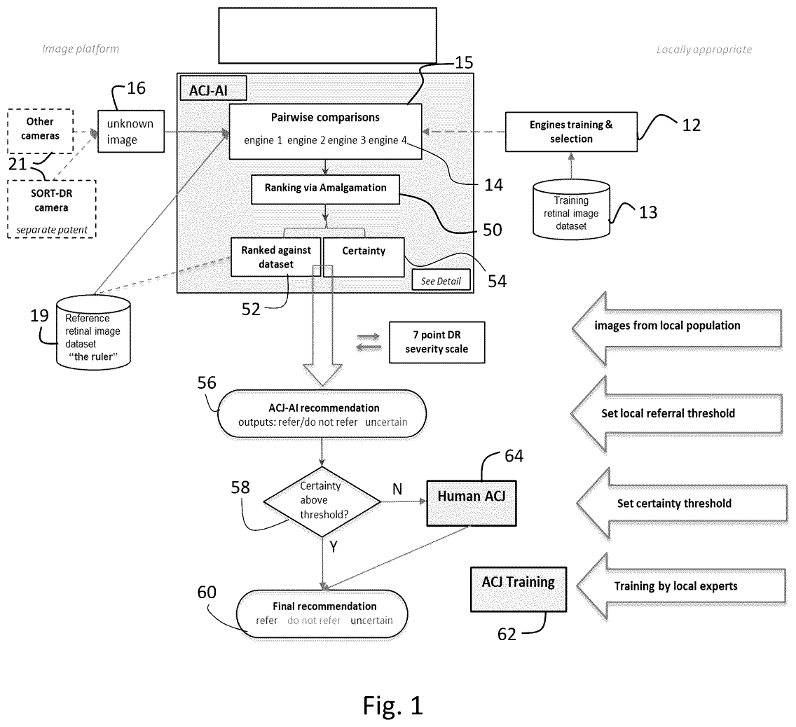

With reference to FIG. 1, the method starts at step 12 by training one or more neural networks using training data sets (in this case the data set is an image set) stored in a database 13. In some embodiments, there are a plurality of neural networks, for example 25 neural networks. The neural networks may be convolutional neural networks.

A plurality of training sets were used to train each of the neural networks. A different training set may be used to train each neural network. The different training sets have been created from a pool of training data sets. The pool of training data sets may vary depending on the disease being classified.

For certain diseases, the diagnosis may be binary, e.g. malignant or benign. In other cases, a more granular assessment is required where the disease is classified by different levels of severity. Different medical authorities may have different ways to grade severity level. For example, there may be five, seven, or another number of different classes of disease severity. Those classifications may be different on a country-by-country, authority-by-authority of state-by-state basis.

A plurality of training sets is generated to train the neural networks. There may be a one-to-one mapping of a training set to a neural network. In this case, there may be 25 training sets and 25 neural networks. The 25 training sets may be constructed by taking samples from a plurality of different image sets. Whilst the number from each of the image sets does not matter, where there are different classifications of disease severity, there are images for each of those classifications within each of the 25 training sets. Augmentation may be used to increase the number of training examples within each training set. For example, augmentations such as flip and rotations of the images may be performed. One advantage of using pairwise comparison is to obtain accurate comparison classification with fewer images. The number of pairs is in a proportion of n(n−1) where n is the number of images, which will be exponentially larger than the number of images. For example, 100 images can derive 9,900 pairs for permutation when the selection order is a factor, or 4950 pairs for combination when the order is not a factor.

Each neural network is trained using its designated training set. By using different training sets, independence of each trained neural network is ensured.

The validation data set may be a different data set.

The neural networks (AI) 14 are trained to perform pair wise comparisons of two images at step 15. One of those images 16 may be an image from a reference set stored in a reference database 19, and the other may be a patient's image. The patient's image may be captured using one or more image capturing devices 21, for example. The one or more image capturing devices 21 in FIG. 1 may include, for example, cameras, X-ray scanners, MRI scanners, ultrasound probes, CT scanners, or mammography X-ray scanners, depending on the disease being investigated. b

The patient's image(s) may be pre-processed to be 512×512 pixels, where the images are two-dimensional (2D) images, e.g. X-rays. The image(s) may be pre-processed to be 256×256×128 pixels, where the images are three-dimensional (3D) images, e.g. MRI. Other sizes are also envisaged, and this particular size is given for illustrative purposes only. The example images of the ruler may also be pre-processed to be of the same size, or at least similar.

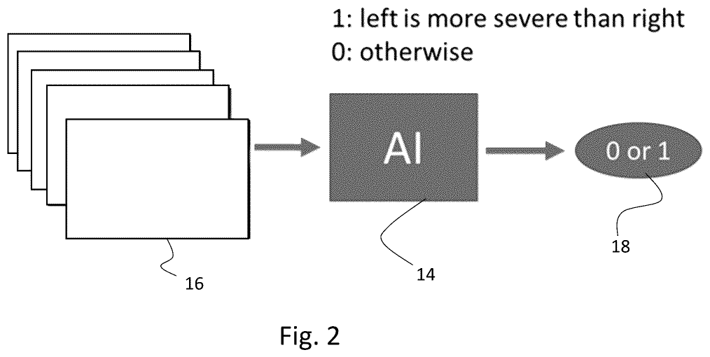

With reference to FIG. 2, an output from the neural networks 14 may be a decision 18 as to whether a first image 16 has more severe disease than a second image 16. The terms first and second may be used interchangeably with the terms left and right, because the images being compared may be positioned side by side. The result may be binary, e.g. 0 or 1, where 1 signifies that the first image has more severe disease than the second image, or vice, versa. In addition, the result may be a probability of the first image having more severe disease than the second image, e.g. the result may be between 0 and 1, where the total equates to 1.

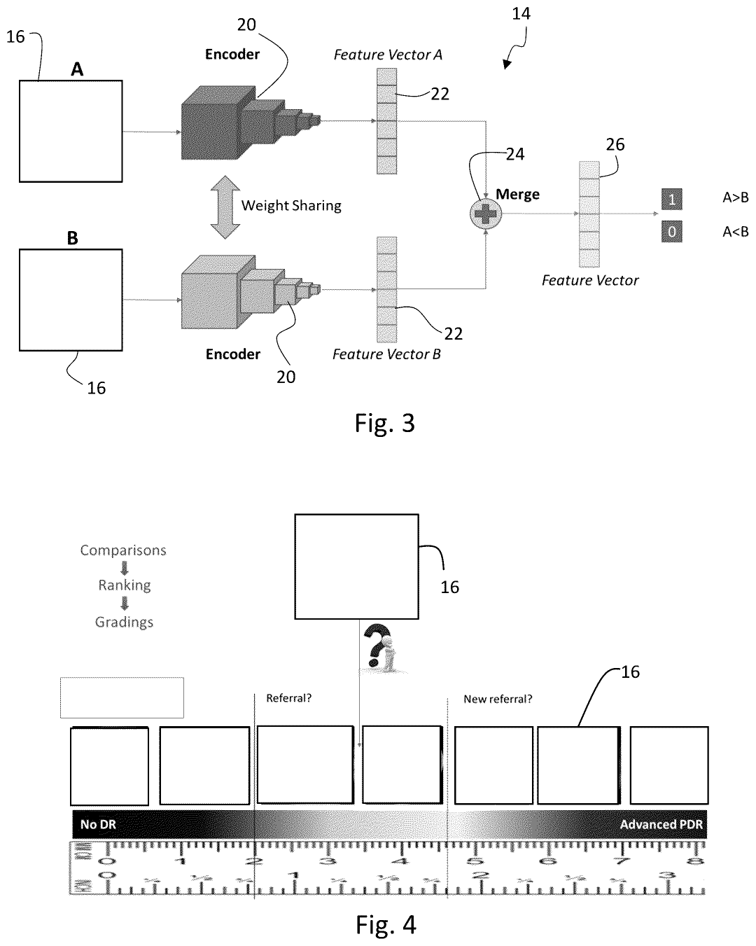

More specifically, with reference to FIG. 3, a pair of medical images A and B may be compared using the neural network 14. Each image 16 may be supplied to an encoder 20. In the context of 2D images, such as X-ray, the encoder may be a 2D encoder. In the context of 3D images, such as MRI images, the encoder may be a 3D encoder. In the context of waveforms, e.g. ECG trace data, the same neural network may be used and the encoder may be modified to be a 1D encoder. The image 16 may be supplied as an input vector of grayscale values in RGB format, for example. The grayscale values may be concatenated together to form the input vector. A series of encoding operations are performed on each image in parallel. The encoding operations include convolutions performed by convolution layers. The encoding operations also include pooling and dropout layers. In this way, the encoder 20 is able to learn features from each of the images 16. The features are constructed as a feature vector 22. There are two feature vectors, A and B, one for each input image being compared. A summing layer 24 is included to sum together the feature vectors 22. The summing layer 24 may perform the addition in various ways, including adding together the individual integers within the feature vectors 22, taking an average value of corresponding integers from the feature vectors, or concatenating the feature vectors together 22. The output from the summing layer 24 is a merged feature vector 26.

The merged feature vector 26 is applied to one or more fully connected layers to obtain a binary result, e.g. 1 or 0, where 1 signifies that the first image has more severe disease than the second image, or vice versa depending on the convention used. The result may also be a probability, e.g. between 0 and 1, as an alternative to being a binary result, e.g. 0 or 1.

The neural networks of the embodiments herein are different from the prior art at least in that they compare severities between a pair of the medical data, in this case medical images (i.e. differences), whereas prior art models such as Siamese and SimCLR strategies learn features via comparing similarities between two images.

It will be appreciated that a binary classification is useful for understanding a comparative severity of disease compared to a reference image. A referral for further investigation is achieved when the reference image is at a threshold for referral. Whilst this has some merit, such thresholds can and often need to change when new medical knowledge is obtained, and depending on referral culture within a local region, and even budgetary constraints where patients may need to wait for more severe disease before being referred. To address this issue, a reference image set 19 is employed which contains a plurality of example images ranked according to their severity of disease, pairwise comparison is performed using the ranked images to slot the patient's image within the ranked list, and a threshold for referral may be applied to the results, as described in more detail below.

The reference data set 19 referred to herein is called a ruler. The ruler may include a plurality of medical data examples. For example, where the medical data includes images, the ruler may include 150 images or another number. This number may change according to specific embodiments but may be found to be suitable for certain diseases. Where the medical data is trace data, there may be 150, or another number, of trace data examples within the ruler. Furthermore, where the medical data is a three-dimensional image, e.g. CT scan images, the ruler may include 150 3D image examples.

Where a disease can be classified in terms of its severity, the images within the ruler may include example images within each class of severity. The number of image examples in each class is selected to be approximately the same, or even the same. The number of images per class is taken to be the number of images from the class which has the least number of example images. The ruler includes its own classes, having more classes than the other standard class systems. There may be several different classes within the ruler. Each of those classes may represent a category of disease severity. The ruler image set includes images within each of the classes.

To obtain the ranking, pairwise comparison is performed using the neural networks to compare each image against every other image in the reference set 19. The images can be ranked depending on how many images are more/less severe than it, e.g. counting the 1s and 0s. In this way, ranking is performed based on the pairwise comparisons.

Referring again to FIG. 1, at step 15, pairwise comparison of the patient's sample image is performed on each image within the ruler. This is performed using each neural network. In some embodiments, the number of neural networks can be reduced to a subset of neural networks. Reducing the number of neural networks to a subset is described in more detail below.

Once the patient's image has been applied to each neural network, a set of results are obtained. When the ruler includes 150 reference images, for example, the results will be 150 binary numbers (e.g. 0 or 1). For example, there may be one hundred 0s, where the patient's image is less severe than the first one hundred example images in the ruler. There may also be 50 1s where the patient's image is more severe than the last 50 images within the ruler. Of course, some comparisons may be uncertain (due noise in data or due lack of features to train the AI algorithm) and there may not be a clear boundary between the sets of 1s and a clear set of 0s, e.g. there could be a middle of the output vector including both 0s and 1s mixed.

Once all neural networks (engines) 14 have provided an output vector, the data is ranked by amalgamation at step 50. At step 52, the result of the amalgamation is ranked against the ruler. In other words, a position within the ruler is obtained for the patient's image. This can be seen more clearly with reference to FIG. 4, which shows how the patient's image 16 is slotted within with ruler's example images 16. A certainty score is obtained at step 54 providing a degree of certainty regarding the position of the patient's image within the ruler.

It is noted that by “slotting” the patient's image within the ruler, the rank of the patient's image with respect to the images in the ruler is obtained. The ruler does not acquire the patient's image as an additional example image. In other words, the ruler will contain the same number of example images before and after the comparison has been performed. However, in some embodiments, the ruler can be augmented offline by adding the patient's image if desired.

In one embodiment, the amalgamation is performed using a group Lasso regularised logistic regression model.

Using the group Lasso regularised logistic regression model, a plurality of thresholds of disease severity are set within the ruler. Each threshold of disease severity is located at a boundary between each class of the ruler.

The Lasso regression model may be trained using cross validation using medical data examples from the ruler to select the optimal regularisation parameter. The cross validation may be 10-fold cross validation. The decision boundary of the group Lasso regularised logistic regression model may be one of the thresholds of disease severity. There may be a plurality of Lasso regression models trained, each having a different decision boundary associated with a different threshold of disease severity. In this way, different Lasso regression models may be used to adjust the severity of the referral threshold. In this way, the threshold of disease severity of the selected Lasso regression model may be considered a threshold for referral.

The output vectors from each of the 25 neural networks may be applied to the Lasso regression model. The best performing neural networks may be selected as a subset for a specific referral threshold. The number of engines within the subset may be decided using statistical measures including, for example cross validation error, prediction error, accuracy, sensitivity, specificity, and area under the receiver operating characteristics curve. By reducing the number of neural networks to a subset, computation time may be reduced in the implementation, and accuracy can be improved.

The output from the group Lasso regularised logistic regression model may be a binary outcome, for example 0 or 1, regarding whether the patient's image should be sent for referral or not, and a predicted probability of the patient's image should be sent for referral. The optimal discrimination rule is derived to separate referral images from non-referrals based on Youden J's statistics (J=sensitivity+specificity−1).

As described above, the output vectors from the neural networks may be used as input for the group Lasso regularised logistic regression model, and there may be some uncertainty regarding the vectors from the neural networks. At step 54, bootstrapping may be used to propagate such uncertainty and to estimate the credible interval for the output predicted probability from the group Lasso regularised logistic regression model. The width of the credible interval may be used to determine a uncertainty value. The uncertainty value may be communicated to the user as a %. The certainty is then 100-uncertainty.

In another embodiment, instead of Lasso regression, the Bradley-Terry-Luce (BTL) model may be used. In this embodiment, no training is required to produce the ranking. Instead, the output vectors from the neural networks 14 (or the subset thereof), are tabulated using a comparison matrix. Each cell of the matrix is an accumulated score of corresponding values from each of the output vectors. The cells of the matrix are compared to a threshold for referral.

In another embodiment, a frequency approach may be used. In such an embodiment, pairwise comparison is performed on the plurality of neural networks, using the ruler image set, and another known image set as the reference image set. The reference images include images with all and different disease severity, and their comparison results against the ruler images are known. The patient's image is compared against all reference images, and a score can be calculated based on the frequency that the reference images are more severe than the patient's image. This score can be used to map to the ruler images, and to determine the rank of the patient's image within the ruler images. A threshold for determining the severity of the patient's image is applied based on the rank and severity of the ruler images One severity class of the patient's image can be determined by output vectors from one neural network. If output vectors from multiple neural networks are used, majority voting can be applied to determine the final severity class of the patient's image.

In another embodiment, a S-curve may be used. This method can be divided into two steps.

The first step is the original S-curve fitting step, which is a regression-based method to produce an estimated score for each patient' image. For each patient's image, one AI pairwise comparison engine gives comparison results against 150 reference images, including 150 0s/1s with the corresponding probabilities for each pair of comparison. One S-curve is then fitted for each patient image to describe the relationship between 150 probabilities from one AI pairwise comparison engine and the normalised score of the ruler images. The score for the patient image is then estimated where the S-curve crosses the 0.5 horizontal line, which corresponds to a probability value of 0.5.

The second step is linear discriminant analysis. It utilises the estimated score from S-curve and amalgamates the score from multiple AI pairwise comparison engines to predict the disease severity for each patient image.

In another embodiment, the various classes of the reference dataset are used as predictors. The results of pairwise comparisons of each image from a reference image dataset when compared with each image of the ruler dataset. The output of the pairwise comparisons is the probability that one image of each pair is more severe than the other. For example, the first image in the reference data is compared with all the images of class one of the ruler dataset. The mean of the probabilities is calculated. Similarly, the means of this first reference image is compared with each of the images of the other classes.

Means of the probabilities from pairwise comparisons: The reference database is divided into two parts based on a pre-determined threshold. For example, if we wished to distinguish between different severities, we would separate the reference grade images below the threshold from the reference images above the threshold. We calculate the average and the standard deviations of the means of all reference images against ruler class below the threshold. Similarly, we calculate the means and standard deviations of each of the other predictors above the threshold.

Training, validation, testing: The means and standard deviations for the dataset is derived from the training dataset for each AI pairwise comparison models during training. Ten folds cross validation were performed to avoid overfitting.

The Prior: is the proportion of reference 012 versus reference 34 in the training, validation and testing dataset.

Likelihood and likelihood ratios: Given the means and standard deviations, we calculate the likelihoods for each image in the reference dataset. Combining the likelihood and prior gives the probability that a given image belongs to reference 012 or alternatively reference 34. The likelihood ratio tells us how many times a given image is more or less likely to be reference 34 than reference 012. In logarithmic form, the likelihood ratio ranges from the positive through zero to negative numbers. The likelihood ratio is used to rank the whole reference image dataset in order of severity.

Cut-offs: Using different cut offs, the sensitivity and specificity, the negative and positive predictive values, the ROC and AUC, the precision and recall were computed. Calibration is performed to determine the “mean, weak and moderate” calibration to describe the behaviour of our model. The frequency, location of errors and their distribution relative to the ranking has been analysed to optimise the negative predictive value of the model.

Amalgamation of Models: This was done in one of two ways. The pairwise comparisons for each of the predictors were simply grouped together and the means calculated. Alternatively, the likelihood ratios were multiplied. Both methods assumed independence amongst AI pairwise comparison engines and amongst different predictors.

The basic method of Naive Bayes is only illustrated above. There are further optimisations. The number of predictors could be made up to sub-class to increase the granularity when needed. All or some of the steps could be used to form new classes. Those with less predictive value could be discarded. Different models could be used for different thresholds.

Referring again to FIG. 1, step 56 corresponds to the output, e.g. binary 1 or 0 regarding referral or no referral, together with the certainty value. At step 58, the certainty value is compared to a certainty threshold. The certainty threshold may be set to provided flexibility depending on the competence of the medical professionals involved. If the certainty value is above the certainty threshold, a final recommendation is provided at step 60. If, at step 58, the certainty value is at or below the certainty threshold, the patient's image is sent to a human operator at step 62. After step 62, the final recommendation may be provided at step 60. The final recommendation may be to refer, do not refer, uncertain. For uncertain cases, the decision will be to refer. In other words, there are two outcomes, e.g. to refer or not to refer.

1. A computer-implemented method of determining if a medical data sample requires referral for investigation for a disease, the method comprising:
performing a pairwise comparison of the medical data sample against each example of medical data in a reference data set using one or more machine learning algorithms to determine a difference in severity of the medical data compared to each example medical data in the reference data set; and
flagging the medical data sample as requiring referral for investigation for the disease based on results of the pairwise comparisons, wherein the reference data set includes a plurality of medical data examples, the plurality of medical data examples ranked according to their degree of severity of disease.

2. The computer-implemented method of claim 1 further comprising:
determining a position of the medical data sample against the medical data examples within the reference data set; and
comparing the position of the medical data sample with a threshold for referral,
wherein the flagging the medical data sample as requiring referral comprises flagging the medical data sample as requiring referral if the position of the medical data sample is above the threshold for referral.

3. The computer-implemented method of claim 1, wherein the one or more machine learning algorithms comprises one or more neural networks.

4. The computer-implemented method of claim 3, wherein the or each neural network is a convolutional neural network.

5. The computer-implemented method of claim 4, wherein the one or more convolutional neural networks is a plurality of convolutional neural networks.

6. The computer-implemented method of claim 5, wherein each of the plurality of convolutional neural networks is trained on a different data set.

7. The computer-implemented method of claim 5, further comprising amalgamating the results of the pairwise comparisons from each convolutional neural network, and wherein the sending the medical data sample for referral for investigation for the disease is based on the amalgamated pairwise comparisons.

8. The computer-implemented method of claim 7, wherein the amalgamating of the results of the pairwise comparisons comprises supplying the results of the pairwise comparison as inputs to one or more lasso regression models, the or each lasso regression model having a decision boundary associated with a threshold of disease severity.

9. The computer-implemented method of claim 8, wherein the or each lasso regression model is a plurality of lasso regression models and wherein the threshold of disease severity for each model is different.

10. The computer-implemented method of claim 7, further comprising estimating a confidence interval of a probability of needing referral.

11. The computer-implemented method of claim 10, wherein the estimating comprises performing bootstrapping.

12. The computer-implemented method of claim 7, wherein the amalgamating the results of the pairwise comparison comprises accumulating results from each convolutional neural network, and comparing the accumulated results to the threshold for referral.

13. The computer-implemented method of claim 7, wherein the amalgamating the results comprises setting a plurality of thresholds of disease severity within the reference image set, and determining a frequency of occurrence of the medical data above each of the thresholds of disease severity.

14. The computer-implemented method of claim 13, wherein the threshold for referral corresponds to one of the plurality of thresholds of disease severity.

15. The computer-implemented method of claim 7, wherein the amalgamating the results of the pairwise comparison comprises fitting an S-curve to the results of each convolutional neural network; determining a probability of requiring referral based each fitted S-curve; and performing linear discriminant analysis on the determined probabilities.

16. The computer-implemented method of claim 6, further comprising selecting a subset of the plurality of convolutional neural networks using a selecting algorithm.

17. The computer-implemented method of claim 16, wherein the selecting algorithm comprises a lasso regression model, the lasso regression model having a decision boundary associated with the threshold for referral, the selecting comprising applying the results from each convolutional neural network into the lasso regression model, ordering the convolutional neural networks in terms of accuracy at predicting referral, and selecting a predetermined number of the highest ranked convolutional neural networks as the subset.

18. The computer implemented method claim 1 further comprising:
receiving the reference data set; and
performing pairwise comparison of each medical data example of the plurality of examples of medical data within the reference data set against every other medical data example of the plurality of medical data example within the reference data set; and
ranking the plurality of medical data examples according to a degree of severity of disease based on the pairwise comparisons.

19. The computer-implemented method claim 1 wherein the disease is selected from a list of diseases including: breast cancer, pneumonia, lung cancer, skin cancer, and cardiovascular disease.

20. The computer-implemented method of claim 1, wherein the medical data is selected from a list of medical data including: a two-dimensional image, a three-dimensional image, and trace data.

21. The computer-implemented method of claim 20, wherein the two-dimensional image comprises X-ray and mammography X-ray, and wherein the three-dimensional image comprises a three-dimensional image selected from a list of three-dimensional images including: a magnetic resonance image, a computerised tomography images, and an Ultrasound image,
and wherein the trace data comprises an eco-cardiogram.

22. A computer-implemented method of ranking a plurality of medical data examples within a reference data set, the method comprising:
receiving the reference data set; and
performing pairwise comparison of each medical data example within the reference data set against every medical data example within the reference data set; and
ranking the plurality of medical data examples according to a degree of severity of disease based on the pairwise comparisons.

23. A non-transitory computer-readable medium including instructions stored thereon that when executed by one or more processors cause the processor to:
perform a pairwise comparison of the medical data sample against each example of medical data in a reference data set using one or more machine learning algorithms to determine a difference in severity of the medical data compared to each example medical data in the reference data set; and
flag the medical data sample as requiring referral for investigation for the disease based on results of the pairwise comparisons,
wherein the reference data set includes a plurality of medical data examples, the plurality of medical data examples ranked according to their degree of severity of disease.
